(19) United States
(12) Patent Application Publication
(10) Pub. No.: US 2023/0032049 A1
(43) Pub. Date: Feb. 2, 2023

(54) SYSTEMS AND METHODS FOR TREATMENT OF SPINAL DEFORMITIES

(71) Applicant: David Skaggs, Los Angeles, CA (US)

(72) Inventor: David Skaggs, Los Angeles, CA (US)

(21) Appl. No.: 17/874,644

(22) Filed: Jul. 27, 2022

Related U.S. Application Data
(60) Provisional application No. 63/227,079, filed on Jul. 29, 2021.

(51) Int. Cl. A61B 17/70

(52) U.S. Cl. CPC A61B 17/70; A61B 17/7032; A61B 17/7044

(57) ABSTRACT

Systems and methods are provided for treating and/or correcting spinal deformities. An implantable spinal system comprises first and second fasteners, such as bone anchors, and a connector element extendible between the first and second bone anchors. The connector element is configured for distraction between the first and second bone anchors on a concave side of the vertebral column. The connector element provides sufficient force to distract the spine to correct the curved portion of the vertebral column. The implantable spinal system may be particularly useful as a distraction and motion preservation system for treatment of scoliosis in a growing child, adolescent or adult. The system may be configured to at least partially correct the curved portion of a spinal column in at least two different planes, such as the frontal plane and/or the sagittal plane, or to correct a rotation in the transverse plane.

### CROSS-REFERENCE TO RELATED APPLICATIONS

This application claims the benefit of U.S. Provisional Application Ser. No. 63/227,079, filed Jul. 29, 2021, the complete disclosure of which is incorporated herein by reference in its entirety for all purposes.

### FIELD

The present disclosure relates to an implantable spinal system and a method of using such a system for the correction of a spinal deformity, and more particularly, to an implantable spinal system and method of using the system for multi-plane correction and/or prevention of the spine curvature of a patient with scoliosis in a fusionless, motion preserving manner.

### BACKGROUND

Scoliosis can generally be described as the abnormal, sideways curvature of the spine in the frontal plane, though it often is accompanied by deformity in the sagittal and transverse planes as well. It can affect people of any age, from babies to adults, but occurs most often during childhood or early adolescence. In most cases, the condition has no known cause, which is why it is often referred to as idiopathic scoliosis. In the most common form of scoliosis, adolescent idiopathic scoliosis, there is decreased kyphosis of the thoracic spine, which can limit the space available for pulmonary function in the thorax, as well as negatively affect the alignment of the cervical and lumbar spine.

Treatments for scoliosis depend on the severity of the condition. For some patients, the use of an external back brace over time may be sufficient to minimize progression of the deformity. The brace may be worn just at night, or all the time. For other patients, due to the severity of the curvature, or when bracing has failed to result in a desired outcome, a surgical approach may be necessary. One of the surgical approaches common today is to perform a spinal fusion surgery. A typical spinal fusion surgery for correction of scoliosis involves implanting rods to realign the spine by securing the rods to the spine using hooks and screws. Over time, the rigid system promotes fusion between adjacent spinal segments secured to the rigid rods, thereby maintaining the corrected curvature by eliminating the ability of the spinal segments to move out of alignment relative to one another.

The spine fusion surgery may be performed using an anterior surgical approach to the thoracic spine in which the surgeon goes through the front of the patient's chest, and specifically through an incision in the side of the chest, to secure the corrective spinal fusion system to the vertebrae. Usually, a lung must be deflated to access the spine during the procedure, and there is risk of damaging organs and blood vessels, and hurting pulmonary function during the implantation process. If the fusion is extended to the lumbar spine, the diaphragm is often cut and released from the chest wall and spine. Access to the upper thoracic and lowest lumbar spine in a way that allows placement of screws and rods in a continuous line is limited by surrounding bony and vascular anatomy, and carries increased risks of vascular injury as well as an inability to place implants in the desired position. In addition, screws in the anterior vertebral body provide less strength of fixation than posterior pedicle screws, and risk unintended loss of fixation both during surgery and post-operatively.

Surgeons have begun to favor a posterior surgical approach to the spine to avoid the aforementioned problems and enable access of the entire thoracic and lumbar spine with equal ease. And while this posterior surgical approach to fusion surgery has gained in popularity because it avoids the drawbacks to the traditional anterior approach and is a familiar and safe approach for surgeons, the overall spinal fusion procedure still has drawback. In particular, an undesirable result of this type of surgery is the very result the surgery is intended to produce: fusion of the spine, which limits growth and motion.

In young patients, there are growth friendly surgeries designed to improve spine deformity while permitting growth, though these have high complication rates, often require multiple surgeries, and generally do not allow normal growth of the spine. One type of growth friendly surgery for scoliosis is vertebral body tethering. In this surgery, through an anterior approach, spine implants are secured to the vertebral bodies on the convex side of the frontal plane curve. Tension is usually placed across the implants that are connected with a flexible cord (tether) which improves the lateral curve of the spine at the time of surgery. In theory, this approach limits growth thorough compression of growth plates on the convex side of the spine, while allowing growth of the concave half of the spine, leading to further improvement of the lateral curve over time with growth. In the sagittal plane, such tensioning at the time of surgery leads to production of kyphosis. Over time, limiting anterior growth of the spine, while permitting posterior growth of the spine, also leads to production of kyphosis.

While this new procedure has shown promise, it has suffered from certain drawbacks. For example, it requires an anterior approach to the spine, has a high rate of complications such as breakage of the tether and over correction, and may require additional anterior surgery through a scarred surgical field that is associated with increased bleeding as well as other complications such as damage to the lung and pulmonary function. In addition, the tether limits growth and in general has been quite disappointing.

What is therefore needed is a corrective spinal system that can sufficiently treat scoliosis, in either a growing or mature spine by correcting the lateral curvature and/or correcting the hypokyphosis generally found in scoliosis. It would also be desirable to provide a system that avoids fusion, preserves motion, and may be implanted using a posterior surgical approach. Ideally, the system can be converted to a posterior fusion, if necessary, through the same surgical approach (no new tissue damage and scar) and use the same pedicle screws that are already in place, thereby minimizing risk, cost, and surgical time.

### SUMMARY

The present disclosure provides systems and methods for treating and/or correcting spinal deformities. The systems and methods are particularly useful in a posterior surgical approach for the multi-plane treatment of scoliosis in a growing child, adolescent or adult. Additionally, the systems and methods can be applied to the front, anterior part of the spine for the correction of spinal curvature abnormalities. In both approaches, the systems and methods avoid fusion to preserve motion of the spine.

According to one aspect, an implantable spinal system for fusionless, motion preserving treatment is provided for correcting a vertebral column having a curved portion with a concave side and a convex side. The system comprises first and second fasteners, such as bone anchors or the like, and a connector element extendible between the first and second bone anchors. The connector element is configured for distracting the first and second bone anchors on the concave side of the vertebral column. The connector element provides sufficient force to distract the spine to correct the curved portion of the vertebral column.

The implantable spinal system may be particularly useful as a fusionless, posterior concave distraction and motion preservation system for the treatment of scoliosis. In some embodiments, the system is configured to at least partially correct the curved portion of a spinal column in at least two different planes. The system may be configured to at least partially correct the curved portion of the vertebral column in the frontal plane and/or the sagittal plane, In addition, or alternatively, the system may be configured to at least partially correct a rotation of the curved portion of the vertebral column in the transverse plane.

The system provides the benefit of allowing multi-plane (i.e., frontal, sagittal and/or transverse plane) correction of the spine curvature. This is achieved with the use of bone anchors that are connected with a flexible, resilient connector element, which anchors provide distraction, while the entire construct (i.e., anchors and connector element) preserves motion.

The connector element may comprise a rod, cord, cable, band, or spring. The connector element may be flexible, resilient or elastic. The connector may provide distraction forces through springs, pneumatics, magnets, motors or other means of producing force, and these distraction elements may be lengthened over time with additional surgery or without surgery. In certain embodiments, the system further comprises a remote actuator for lengthening the distraction elements from outside of the patient's body. For example, magnetically controlled connecting elements can be lengthened between vertebrae by an external controller.

In some embodiments, an outer sheath for placement over the connector element may be provided. This outer sheath can provide distraction in an elastic or otherwise mobile fashion.

In some embodiments, the bone anchors are specifically configured for placement on the concave side of a curved portion of the spinal column. In one such embodiment, the first bone anchor comprises a first head portion and a first shank portion, and the second bone anchor comprises a second head portion and a second shank portion. The first and second shank portions are configured to engage bone to secure the first and second bone anchors to first and second vertebral bodies on the concave side of the spinal column. The bone anchors may be pedicle screws in some embodiments.

In certain embodiments, the bone anchors comprise pedicle screws configured for securing to vertebral bodies in the spinal column. In other embodiments, the bone anchors comprise posterior anchors configured for securing to ribs or other bones in the patient.

The connector element is configured to be longitudinally displaceable through the first and second head portions. The system may further comprise a securing element, such as a screw, fastener or the like, receivable within the second head portion to secure a portion of the connector element from longitudinal displacement relative to the first and second head portions. In one embodiment, the securing element comprises a screw and the second head portion comprises an internal threaded portion threadably engageable with the screw.

In some embodiments, the system further comprises a distraction member configured to couple to one portion of the connector element and to maintain a force on the connector element between the first and second bone anchors. The distraction member may be configured to couple to the proximal end of the connector element adjacent to a side of the first head portion facing away from the second bone anchor. In certain embodiments, the distraction member comprises an external instrument configured to apply a force to the connector element. In other embodiments, the distraction member may comprise an internal distraction device, such as a coil spring surrounding the connector element.

According to another aspect, a method for correcting a curved portion of a vertebral column in a fusionless, motion preserving manner is provided. The method comprises advancing first and second bone anchors through an opening in the patient to first and second vertebral bodies within the curved portion of the vertebral column. The first and second bone anchors are secured to the first and second vertebral bodies. A connector element is positioned between the first and second bone anchors to distract the vertebral column and at least partially correct the curved portion. The connector element provides sufficient force to distract the spine to correct the curved portion of the vertebral column.

In one embodiment, the opening is a posterior opening in the back of the patient, and the first and second bone anchors are secured to the curved portion of the posterior side of the vertebral column. In another embodiment, the opening is an anterior opening in the front of the patient, and the first and second bone anchors are secured to the curved portion of the anterior side of the vertebral column.

In some embodiments, the curved portion of the vertebral column has a concave side and a convex side. The method further comprises securing the first and second bone anchors to the concave side of the vertebral column. A force is applied to the connector element to distract the vertebral column on the concave side to correct the curved portion.

In certain embodiments, the bone anchors may be secured to vertebral bodies in the spinal column. In other embodiments, the bone anchors may be secured to ribs or other bones in the patient.

In some embodiments, the method further comprises increasing a length of the connector element to increase the distraction applied to the spinal column and further correct the deformity. For example, the length of the connector element may be increased gradually over time, in discrete steps or continuously, to gradually change the curvature of the spinal column. In certain embodiments, the connector element is lengthened by an external actuator or controller located outside of the patient's body.

In some embodiments, the method comprises advancing a first end of the connector element through first and second head portions of the first and second bone anchors, respectively, such that the connector element extends at least from the first bone anchor to the second bone anchor. A first end of the connector element may be secured to the first head portion. A force may then be applied to a second end of the connector element from the second head portion to achieve the desired distraction to the curved portion of the spinal column. The connector element is then secured to the second head portion to maintain the distraction between the first and second bone anchors. Alternatively, the connector element may be at least partially secured to the first and second bone anchors and then distracted to achieve the desired force. The steps above may be repeated at either an adjacent, or different, level of the same spinal column.

In some embodiments, the method includes distracting the connector element sufficiently to at least partially correct the curved portion of a spinal column in at least two different planes. The connector element may be distracted sufficiently to at least partially correct the curved portion of the vertebral column in the frontal plane and/or the sagittal plane, In addition, or alternatively, the connector element may be distracted sufficiently to at least partially correct a rotation of the curved portion of the vertebral column in the transverse plane.

The connector element may comprise a rod, cord, cable, band, or spring. The connector element may be flexible, resilient or elastic. In some embodiments, an outer sheath may be positioned over the connector element.

In some embodiments, the first bone anchor comprises a first head portion and a first shank portion, and the second bone anchor comprises a second head portion and a second shank portion. The first and second shank portions are configured to engage bone to secure the first and second bone anchors to first and second vertebral bodies. The bone anchors may be pedicle screws in some embodiments.

The connector element may be longitudinally displaced through the first and second head portions. The method may further comprise securing the connector element to the head portions with a screw, fastener or the like that is receivable within the head portions. In one embodiment, a screw is rotated within threads in the head portion to tighten the screw against the connector element.

According to another aspect, a spinal system for correcting a curved portion of a vertebral column comprises a surgical instrument having a distal end configured for advancement through an opening in a patient. The system further includes a distraction device comprising first and second bone anchors configured for removable attachment to the distal end of the surgical instrument. A connector element is extendible between the first and second bone anchors, and a distracting element is configured to apply distraction to the connector element between the first and second bone anchors to distract the curved portion of the vertebral column.

In one embodiment, the opening is a posterior opening in the back of the patient, and the first and second bone anchors are configured to be secured to the curved portion of the posterior side of the vertebral column. In another embodiment, the opening is an anterior opening in the patient, and the first and second bone anchors are configured to be secured to the curved portion of the anterior side of the vertebral column.

In some embodiments, the curved portion of the vertebral column has a concave side and a convex side. The surgical instrument is configured to secure the first and second bone anchors to first and second vertebral bodies on the concave side of the vertebral column.

In some embodiments, the system further comprises a distracting instrument having an opening for receiving one end of the connector element and a distracting mechanism for applying distraction to the connector element. The distracting instrument may be configured to generate sufficient force in the connector element to at least partially correct the curved portion in at least two different planes. In one aspect, the distracting instrument may be configured to generate sufficient force in the connector element to at least partially correct the curved portion of the vertebral column in the frontal plane. In another aspect, the distracting instrument may be configured to generate sufficient force in the connector element to at least partially correct the curved portion of the vertebral column in the sagittal plane. In still another aspect, the distracting instrument may be configured to generate sufficient force in the connector element to at least partially correct a rotation of the curved portion of the vertebral column in the transverse plane.

In certain embodiments, the system may also include an overtube configured for advancing through one or more openings in the patient. The overtube may comprise an internal lumen sized to receive the connector element, and have a rigidity that is greater than a rigidity of the connector element.

In another embodiment, the system may be used to distract across the concavity of the lumbar spine to create lordosis. This can be achieved by implanting the system along the concave portion of the scoliotic curve in the front, anterior part of the spine. Placement of the system in this manner enables distraction in the frontal plane, and derotation of the spine, thus creating lordosis.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 illustrates an implantable distraction device for treating a spinal disorder;

FIG. 2 illustrates a fixation element of the distraction device of FIG. 1;

FIGS. 3A and 3B are representative drawings of a spine having a deformity before and after correction with the implantable spinal system of the present disclosure, in which FIG. 3A shows the spine before correction and FIG. 3B shows the spine after correction;

FIGS. 4A and 4B are representative drawings of a spine having hypokyphosis, illustrating before and after correction of the hypokyphosis in which FIG. 4A shows the spine after correction and FIG. 4B shows the spine before correction; and

FIG. 5 is a representative drawing of the system of FIG. 1 applied to a front, anterior part of a patient's spine.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

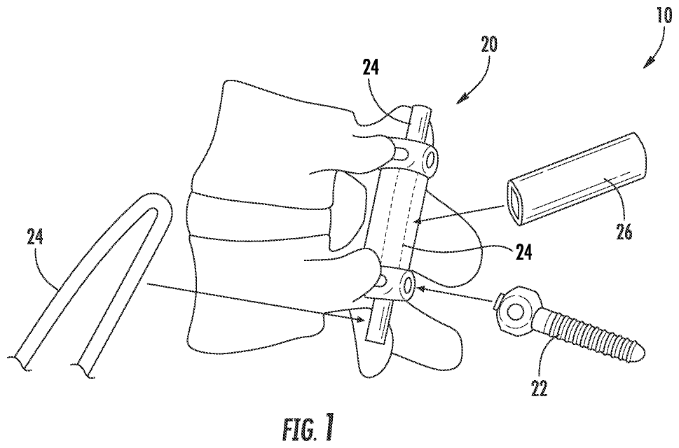

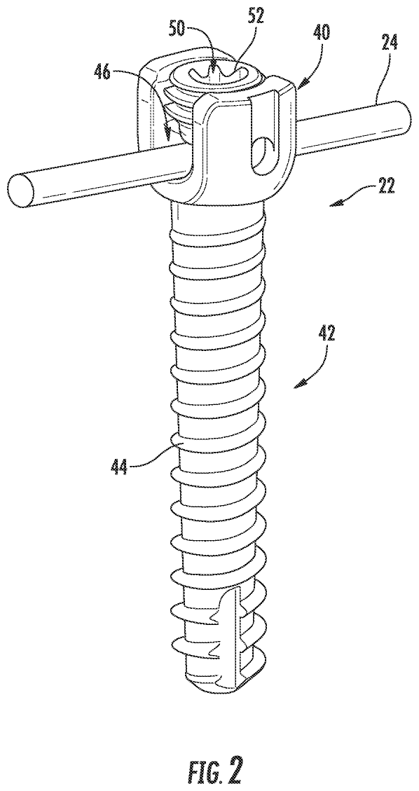

Referring now to FIGS. 1 and 2, an implantable spinal system 10 is provided that may be used to correct spinal deformities, such as a curved portion of a vertebral column. System 10 may be particularly useful for treating and/or correcting scoliosis, kyphosis or other deformities with or without fusing one or more intervertebral bodies. In one embodiment, system 10 is configured to provide posterior concave distraction and motion preservation for scoliosis treatment, and is particularly applicable in a young patient with a growing spine. In another embodiment, the system 10 can be applied to the front, anterior part of the spine.

System 10 includes one or more of distraction devices 20 for the treatment of a spinal segment within a curved portion of a vertebral column. Each distraction device 20 may comprise a pair of fixation devices, such as posterior bone anchors 22. Bone anchor(s) 22 may comprise any anchor configured for attachment to the posterior bone of the spine or ribs, including but not limited, to screws, hooks, darts, ties, or any other element for fixing the longitudinal portion to bone.

In one embodiment, posterior bone anchors 22 comprise pedicle screws that are specifically configured for placement along the concave side of a scoliotic curve. Thus, the pedicle screws are configured to be concave and placed against the spine. Each distraction device 20 allows for correction of the curvature of the spinal segment to which it is attached. As can be seen in the figures and in the description herein, this result is achieved with the use of bone anchors 22 that are secured to the spine and connected with a flexible, resilient connector element 24, which anchors provide distraction, and while the entire construct (i.e., anchors and connector element) allows motion. It is believed that distraction using this posterior system can at least partially correct kyphosis, and improve the sagittal plane deformity typically found in adolescent idiopathic scoliosis in the thoracic spine.

In one such embodiment, bone anchors 22 comprise a pedicle screw having a head portion 40 and a shank portion 42 having a threaded shaft 44 (see FIG. 2). In certain embodiments, bone anchor 22 may comprise elements other than a threaded shaft for securing to a vertebral body, such as vertebral hooks. Threaded shaft 44 may be cannulated or uncannulated. For example, in one embodiment, the bone anchor 22 comprises a cannulated hydroxyapatite-coated vertebral bone screw and the shaft 44 can be cannulated and hydroxyapatite-coated. Head portion 40 may have any suitable cross-sectional shape, such as circular, square, rectangular, polygonal, elliptical or the like. In one embodiment, head portion 40 comprises a substantially U-shaped element (e.g., a tulip-head) having a central channel 46 for receiving connector element 24. Channel 46 may have a width or internal dimension that is substantially the same as an outer diameter of connector element 24. Alternatively, the width of channel 46 may be slightly larger or substantially larger than the diameter of connector element 24.

Head portion 40 may include a threaded region (not shown) for receiving a set screw 50 or other locking element. Set screw 50 includes a mating feature 52, such as a hexalobe interface or the like, for mating with an instrument to rotate set screw relative to bone anchor 22. In some embodiments, set screw 50 threadably engages head portion 40 and secures connector element 22 within channel 46. In other embodiments, connector element 24 may pass through channel 46 without being secured within channel 46.

In other embodiments, head portion 24 may have other cross-sectional shapes, such as circular, square, rectangular, polygonal, elliptical or the like. In these embodiments, head portion 40 may further include an opening, such as a bore, extending from the top surface of head portion 40 to a horizontal through hole that serves the same function as channel 46.

System 10 may further include an introducer (not shown) for implanting bone anchors 22 into the vertebral bodies. In certain embodiments, the introducer includes a screwdriver assembly having a ratcheting handle with a tap for creating a hole in the vertebral body to receive bone anchor 22. The screwdriver may further comprise a distal end that couples to head portion 40 of bone anchor 22 for screwing bone anchor into the hole created by the tap. The hole may be created, for example, within the central hole of an implanted anchor 11.

Connector element 24 is placed between the pedicle screws 22, and together, forms each distraction device 20 of system 10. Distraction is produced and maintained between the screws 22. The connector element 24 may provide distraction forces through springs, pneumatics, magnets, motors or other means of producing force.

In still another aspect of the spinal system, the distraction device 20 may be configured to be shortened or lengthened by surgical or non-surgical methods. A non-surgical method may involve magnets along the length of the device, which can be controlled remotely outside of the patient's body.

System 10 may further comprise a controller or actuator (not shown) coupled to connector element 24 and configured to increase or decrease a length of the connector element 24 between the bone anchors 22. For example, the connector element 24 may be increased in length gradually over time to further correct the spinal deformity. This increase may occur in discrete steps or continuously. In certain embodiments, the controller may be an external controller that remotely controls the length of connector element 24 between adjacent vertebrae. For example, connector element 24 may include one or more magnetic poles and the external controller may comprise an energy source for generating a magnetic field that causes the magnetic poles to move away from each other.

Connector element 24 is configured to provide sufficient resistance between the screws; however, it is also recognized that some distraction can lead to increased motion. The connector element 24 may be formed of a braided or woven polymeric material that also allows some degree of movement to preserve motion. In one embodiment, the connector element 24 may be configured as a flexible but resilient, elastic rod or cord connecting the two screws 22 (FIG. 2). In other embodiments, the connector element 24 may be pleated, or folded, to allow for elongation as the distance between the screws increases with the growth of the spine. In still other embodiments, the connector element may be a flat, elastic band.

While the connector element 24 is shown having a circular cross-section, it may have any cross-section desired such as, but not limited to, square, rectangle, polygonal or elliptical. In one embodiment, connector element 24 may be formed from polyethylene-terephthalate (PET), although it will be recognized that various other materials suitable for implantation within the human body may be used. For example, connector element may comprise other materials, such as metal, polymeric materials or combinations of flexible materials. Connector element 24 may be of any length necessary to extend through the curved portion of the spinal column, for example, between two, three, four or more vertebral bodies of the spinal column.

In some embodiments, connector element 24 may vary in flexibility and elongation properties along the length of the connector element 24. For example, a portion of connector element 24 may be significantly more rigid if greater correction of a spinal deformity is needed at particular levels of the spine and less rigid in levels of the spine requiring less correction. In some embodiments, system 10 may further comprise an internal member, such as a spring member or the like, to provide force to the connector element 24 and potential elongation of the connector element 24. For example, the internal member may be a helical spring, or a polymeric spacer loaded in decompression and surrounding at least a portion of connector element 24.

In other embodiments, the system may comprise an external element or instrument (not shown) that applies force to distract connector element 24. The external instrument may, for example, comprise a handle having an opening for receiving one end of connector element 24. The handle may include a force applying mechanism, such as a rack and cleats, and a user adjustable element, such as a knob, trigger or the like, to pull connector element into the handle and thereby provide force to the connector element that has been placed within bone anchors 22. The mechanism may also include a visual indicator or gauge that provides an indication of the force applied to connector element.

In some embodiments, the connector element 24 can have an outer sheath 26, such as for example, an over-sized polycarbonate urethane (or similar material) outer sheath. In other embodiments, the outer sheath may be an expandable (e.g., pleated) bellows or a telescoping sheath that allows for elongation. This outer sheath can provide distraction in an elastic or otherwise mobile fashion. The outer sheath 28 may also serve as a spacer.

In other embodiments, the system may include an external tube (not shown) that is more rigid than connector element 24. Connector element 24 may, for example, be advanced through the tube to facilitate insertion of connector element 24 through an opening in the patient to the spinal column.

According to another aspect of the spinal system, the distraction device 20 may be formed as a flexible, resilient spring. This spring may also be used with an outer sheath as described above with the connector element 24. The spring may be polymeric or metallic.

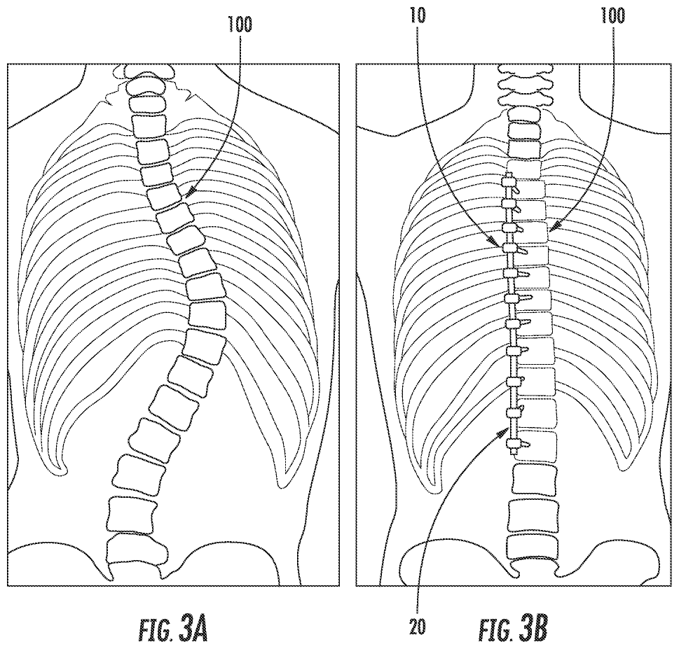

Implantable spinal system 10 may include a series of the distraction devices 20 that may be used along one side of a curved portion of the spine. For example, two or more distraction devices 20 may be linked or “stacked” at discrete or adjacent spinal segments for a modular approach to correct the spinal curvature at that side of the spine. In one embodiment, system 10 is positioned on a concave side 32 of a vertebral column 100 (see FIGS. 3A and 3B). In certain embodiments, the same or a different amount of distraction devices may be used on the opposing side of the spine, as a countereffect, or to supplement, the treatment. The modularity of the spinal system of the present disclosure allows the surgeon to customize the level of distraction at discrete locations on the spine.

System 10 may be inserted using a posterior surgical approach. In one embodiment, a spinal instrument (not shown) may be advanced through a posterior opening in a back of the patient. The spinal instrument may have a distal end that is removably coupled to a first posterior bone anchor 22. The first posterior bone anchor 22 may be secured to a first vertebral body or to a first rib bone. The instrument may then be removed from the patient and reinserted to secure a second posterior bone anchor 22 to a second vertebral body or a second rib bone. This process may be continued until a posterior bone anchor is secured to each vertebral body or rib in the curved portion of the vertebral column. Alternatively, the bone anchors may be secured to only some of the vertebral bodies (e.g., every other vertebral body, or some other suitable pattern).

Once the bone anchors 22 are secured to the vertebral bodies, connector element 24 may be advanced longitudinally through channels 46 in each bone anchor 22. Connector element 24 may be inserted through an extension spring tube (not shown) prior to insertion onto channels 48. A cord alignment instrument (not shown) may be used to align connector element 24 with channels 46. A suitable distraction force may be provided to connector element 24 with an external instrument or an internal member. In some embodiments, the distraction force may be applied to connector element 24 after it has been advanced through all of the bone anchors 22. In other embodiments, the distraction force may be applied to connector element 24 separately between each adjoining vertebral bodies prior to advancing it through the next vertebral body.

The distraction force may be applied to connector element 24 sequentially one motion segment at a time, or the distraction force may be applied to more than one motion segment at the same time. Distraction will provide an initial correction of the curve being treated, but more importantly it will allow for growth modulation at the levels instrumented. The amount of distraction will vary from patient to patient and ultimately be dependent on a multitude of factors, including preoperative Cobb angle, curve flexibility, curve type(s), curve location(s), skeletal maturity and anticipated growth among others. The forces applied to the different levels should be selected such that distraction and the resulting growth modulation will be able to achieve the desired correction over time.

Once connector element 24 has been distracted to a prescribed force to adequately correct the curvature, it may be secured to each of the bone anchors 22. Alternatively, connector element 24 may be partially or fully secured to one or more of the bone anchors 22 before the distraction step. In one embodiment, set screws 50 are placed into head portions 40 of bone anchors 22 and screwed into the threaded portions of head portions 40 to secure connector element 24 to each bone anchor 22. Screws 50 may be secured to bone anchors 22 with, for example, a T-handle screwdriver or the like. In some embodiments, connector element 24 may only be secured to some of the bone anchors 24. If there is excess length of connector element 24 present, it may be trimmed before or after distraction. The distraction on connector element 24 may be adjustable by, for example, a spring member or other distraction device to attain the desired amount of distraction in connector element 24.

The placement of system 10 may depend on the type of deformity to be corrected and/or the curvature of the spinal column to be corrected. For example, the position of bone anchors 22 may be dictated by the curvature of the spine on a case by case basis. In some instances, the position of each bone anchor 22 may vary from one vertebra to the next vertebra (or one rib to the next). In instances where the spinal column has a compound curvature (e.g., has multiple curved portions), it may be desirable to implant one or more systems 10 on the concave sides of each of the curved portions of the spinal column.

A single correction system 10 may be implanted and applied to the spinal column or multiple correction systems 10 may be applied to the spinal column. For example, multiple correction systems 10 may be implanted and applied in parallel on a single aspect of the spinal column and/or multiple correction systems 10 may be implanted at different locations of the spinal column (e.g., throughout different curved regions of the spine, and/or at different levels of the spine). In certain embodiments, a first correction system 10 may be implanted on the concave side of the curvature, and a second correction system 10 may be implanted on the convex side of the curvature.

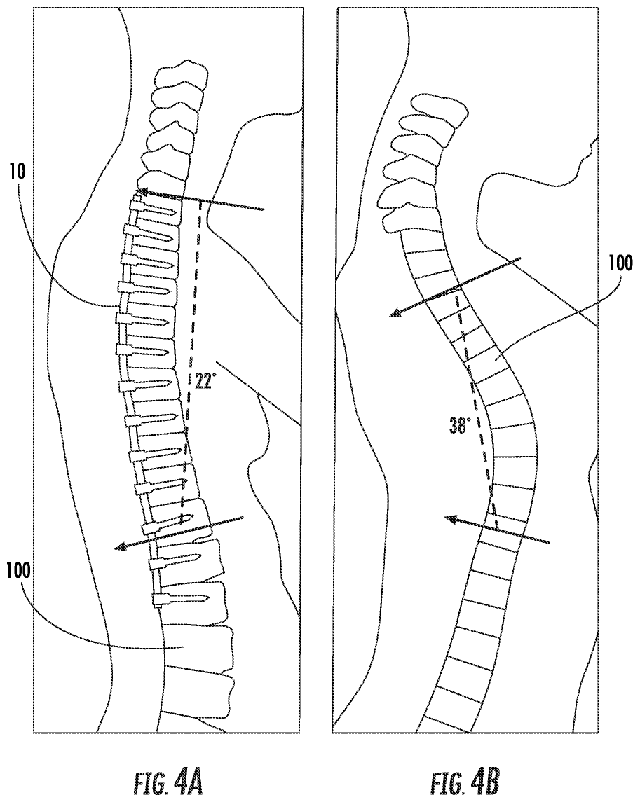

System 10 is particularly advantageous for correcting abnormal curvatures caused by scoliosis. In one embodiment, distraction of scoliosis is achieved across concave pedicle screws. As previously discussed, one of the benefits of the present system is that it allows for multi-plane correction of the scoliotic spine. The system improves scoliosis in the coronal plane, and/or improves hypokyphosis in the sagittal plane. FIG. 3A illustrates a spinal column 100 before such correction, and FIG. 3B illustrates the spinal column 100 after system 10 has been implanted and corrected the abnormal curvature in the spinal column 100. FIG. 4B illustrates a spinal column 100 with hypokyphosis (e.g., about 38 degrees of kyphosis) and FIG. 4A illustrates the spinal column 100 after correction with the device(s) and methods described herein (e.g., about 22 degrees of kyphosis).

The system may also improve rotation in the transverse plane. As it provides mild distraction, the system allows for increased intervertebral motion in all planes. Without such movement being allowed, concave facet joints in a scoliotic spine would lose motion and auto-fuse over time. Additionally, because the system is configured to preserve motion, it can be converted to a posterior spinal fusion, if necessary, through the same incision, using the same screws, if the mild distraction does not provide the desired results.

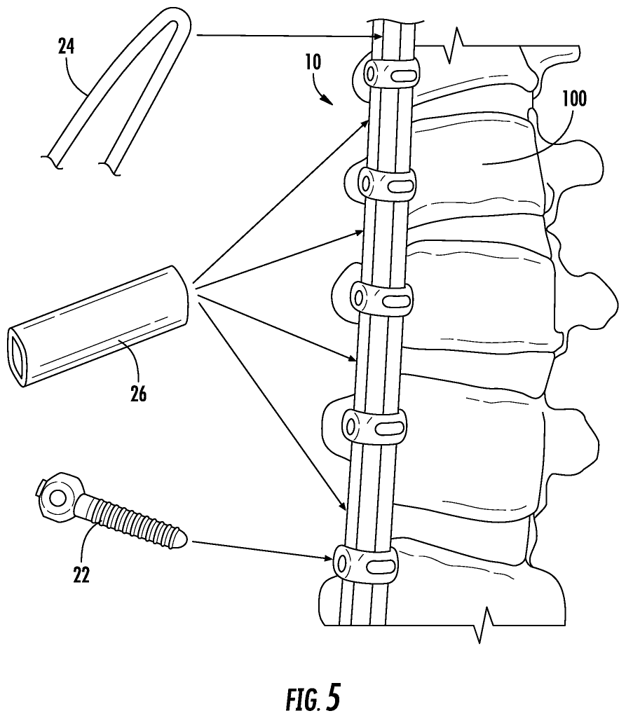

In another embodiment, the system 10 may be used to distract across the concavity of the lumbar spine to create lordosis. For example, as show in FIG. 5, the system 10 can be applied to a front, anterior part of a patient's spine. The system 10 can be placed along the concave portion of the scoliotic curve in the front, anterior part of the spine. Placement of the system 10 in this manner enables distraction in the frontal plane, and derotation of the spine, thus creating desirable lordosis. In the lumbar spine, creation of lordosis provides a clinical benefit. The application of the present system 10 in the front (anterior) part of the spine provides a distinct advantage over currently existing spinal systems that compress across the convex lumbar spine, reducing desired lordosis of the spine, while creating undesired kyphosis instead.

As noted above, one of the great benefits of the spinal system of the present disclosure is that fusion is avoided and motion is preserved, which is particularly desirable for a young patient having a growing spine. This treatment should make the spine and patient taller. Of course, it should be understood that the present spinal system is not limited to use in young patients, as the benefits of the system may be enjoyed by adults with scoliosis as well. For example, the system is particularly desirable for use in adults without a growing spine, but who wish to delay fusion as well.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. An implantable spinal device for correcting a vertebral column having a curved portion with a concave side and a convex side, the device comprising:
first and second bone anchors;
a connector element extendible between the first and second bone anchors; and
wherein the connector element is configured for distracting the first and second bone anchors on the concave side of the vertebral column.

2. The implantable spinal device of claim 1, wherein the connector element comprises a rod, cord, cable, band, or spring.

3. The implantable spinal device of claim 1, further including an outer sheath for placement over the connector element.

4. The implantable spinal device of claim 1, wherein the first and second bone anchor are shaped and configured for placement against first and second vertebral bodies on the concave side of the vertebral column.

5. The implantable spinal device of claim 4, wherein the first bone anchor comprises a first head portion and a first shank portion, and the second bone anchor comprises a second head portion and a second shank portion, the first and second shank portions being configured to engage bone to secure the first and second bone anchors to first and second vertebral bodies.

6. The implantable spinal device of claim 4, wherein the connector element is configured to be longitudinally displaceable through the first and second head portions.

7. The implantable spinal device of claim 5, further comprising a securing element receivable within the second head portion to secure a distal end of the connector element from longitudinal displacement relative to the second head portion.

8. The implantable spinal device of claim 7, further comprising a distraction member configured to couple to a proximal end of the connector element, and configured to maintain a force on the connector element between the first and second bone anchors.

9. The implantable spinal device of claim 1, wherein the system is configured to at least partially correct the curved portion of the vertebral column in the frontal plane or the sagittal plane.

10. The implantable spinal device of claim 1, wherein the system is configured to at least partially correct a rotation of the curved portion of the vertebral column in the transverse plane.

11. A method for correcting a curved portion of a vertebral column in a patient, the method comprising:
advancing first and second bone anchors through an opening in the patient to the curved portion of the vertebral column;
securing the first and second bone anchors to the first and second bones adjacent to, or near, the curved portion of the vertebral column;
positioning a connector element between the first and second bone anchors; and
applying a force to the connector element to distract the vertebral column and at least partially correct the curved portion.

12. The method of claim 11, wherein the curved portion of the vertebral column has a concave side and a convex side, the method further comprising securing the first and second bone anchors to the concave side of the vertebral column.

13. The method of claim 12, further comprising distracting the vertebral column on the concave side to correct the curved portion.

14. The method of claim 13, further comprising advancing a first end of the connector element through first and second head portions of the first and second bone anchors, respectively, such that the connector element extends at least from the first bone anchor to the second bone anchor.

15. The method of claim 14, further comprising securing a first end of the connector element to the first head portion and applying a force to a second end of the connector element.

16. The method of claim 14, further comprising securing the connector element to the second head portion to maintain the force between the first and second bone anchors.

17. The method of claim 14, further comprising generating sufficient force in the connector element to at least partially correct the curved portion in at least two different planes.

18. The method of claim 11, wherein the opening is a posterior opening in the back of the patient, and the first and second bone anchors are secured to the curved portion of a posterior side of the vertebral column.

19. The method of claim 11, wherein the opening is an anterior opening in the patient, and the first and second bone anchors are secured to the curved portion of an anterior side of the vertebral column.

20. A spinal system for correcting a curved portion of a vertebral column, the system comprising:
a first surgical instrument having a distal end configured for advancement through an opening in a patient;
first and second bone anchors configured for removable attachment to the distal end of the surgical instrument;
a connector element extendible between the first and second bone anchors; and
a second surgical instrument configured to apply a force to the connector element between the first and second bone anchors to distract the curved portion of the vertebral column.

21. The system of claim 20, wherein the curved portion of the vertebral column has a concave side and a convex side, wherein the first surgical instrument is configured to secure the first and second bone anchors to first and second vertebral bodies on the concave side of the vertebral column.

22. The system of claim 20, wherein the second surgical instrument is configured to generate a sufficient force in the connector element to at least partially correct the curved portion in at least two different planes.

23. The system of claim 20, wherein the opening is a posterior opening in the back of the patient, and the first and second bone anchors are configured to be secured to the curved portion of a posterior side of the vertebral column.

24. The system of claim 20, wherein the opening is an anterior opening in the patient, and the first and second bone anchors are configured to be secured to the curved portion of an anterior side of the vertebral column.
